(19) United States
(12) Patent Application Publication
(10) Pub. No.: US 2009/0018509 A1
(43) Pub. Date: Jan. 15, 2009

(54) Cartridge liquid injector

(75) Inventors: Nobuhiro Takahashi, Hanamaki-shi (JP); Hidenori Takahashi, Osaka (JP); Minoru Aihara, Osaka (JP)

Correspondence Address: BIRCH STEWART KOLASCH & BIRCH, PO BOX 747, FALLS CHURCH, VA, 22040-0747, US

(73) Assignees: ICOMES LAB Co., Ltd. Research and development center by collaboration of Morioka city and Iwate; Nipro Corporation

(21) Appl. No.: 12/213,334

(22) Filed: Jun. 18, 2008

(30) Foreign Application Priority Data
Jul. 10, 2007 (JP) 2007-180933

(51) Int. Cl. A61M 5/142

(52) U.S. Cl. 604/187

(57) ABSTRACT

A cartridge liquid injector includes a drive motor rotatable in forward and reverse directions, for applying driving force, a reduction portion reducing a speed of rotational output of the drive motor, a linear motion portion converting rotational output of the reduction portion to linear motion, a plunger pushed forward or pulled back by the linear motion portion to move forward an injection plug of a cartridge, and a reduction ratio switching portion switching a reduction ratio of the reduction portion such that the reduction ratio in pulling back the plunger is lower than the reduction ratio in pushing forward the plunger.

This nonprovisional application is based on Japanese Patent Application No. 2007-180933 filed with the Japan Patent Office on Jul. 10, 2007, the entire contents of which are hereby incorporated by reference.

### FIELD OF THE INVENTION

The present invention relates to a cartridge liquid injector.

### DESCRIPTION OF THE BACKGROUND ART

Generally, a cartridge liquid injector is heavily used as a liquid injector for an injection, with a needle being attached to a tip end opening of a cartridge.

A conventional electrically-driven injection device of such a type includes a pinion turned by a motor with reduction device through a power transmission gear mechanism and a plunger having a rack engaged with the pinion. For example, in injecting an injection into oral cavity, the plunger is moved forward at an extremely low speed. In addition, after an injection operation is completed, rotation of the motor with reduction device is reversed so as to move backward the plunger.

Returning the plunger by reversing the rotation of the motor with reduction device, however, takes too long a time. Therefore, the plunger and a drive mechanism for the same are disconnected from each other so that the plunger can freely be pushed back with hands (see, for example, Japanese Patent Laying-Open No. 2001-070444).

The conventional device, however, gives rise to a problem of insanitation because the plunger is returned with hands, and development of a cartridge liquid injector in which the plunger can quickly be returned by being electrically driven has been demanded.

### SUMMARY OF THE INVENTION

The present invention was made in view of the above, and an object of the present invention is to provide a cartridge liquid injector in which a plunger can quickly be returned by being electrically driven.

In order to achieve the object above, a cartridge liquid injector according to the present invention includes: a drive motor rotatable in forward and reverse directions, for applying driving force; a reduction portion reducing a speed of rotational output of the drive motor; a linear motion conversion portion converting rotational output of the reduction portion to linear motion; a plunger pushed forward or pulled back by the linear motion conversion portion, for moving forward an injection plug of a cartridge; and a reduction ratio switching portion switching a reduction ratio of the reduction portion such that the reduction ratio in pulling back the plunger is lower than the reduction ratio in pushing forward the plunger.

With such an arrangement, according to the present invention, the reduction ratio switching portion can switch the reduction ratio of the reduction portion such that the reduction ratio in pulling back the plunger is lower than the reduction ratio in pushing forward the same. Thus, a speed of pushing forward the plunger can be low and a speed of pulling back the plunger can be high.

In addition, in the cartridge liquid injector according to the present invention, the reduction ratio switching portion is formed to switch the reduction ratio by switching an engaged state of gears in a reduction gear group forming the reduction portion.

With such an arrangement, according to the present invention, the reduction ratio can appropriately and readily be switched by switching an engaged state of the gears in the reduction gear group.

In addition, in the cartridge liquid injector according to the present invention, the reduction ratio switching portion is formed to switch the reduction ratio in response to attachment and removal of the cartridge to and from an injector main body.

With such an arrangement, according to the present invention, the speed of the plunger can automatically be set to an appropriate speed as a result of switching of the reduction ratio by the reduction ratio switching portion in response to attachment and removal of the cartridge to and from the injector main body.

In addition, in the cartridge liquid injector according to the present invention, the drive motor is formed such that rotation in forward and reverse directions thereof is switched in response to attachment and removal of the cartridge to and from an injector main body.

With such an arrangement, according to the present invention, the plunger can appropriately be pushed forward or pulled back as a result of switching between rotations in forward and reverse directions of the drive motor in response to attachment and removal of the cartridge to and from the injector main body.

Thus, according to the cartridge liquid injector of the present invention, the plunger can quickly be pulled back by being electrically driven.

The foregoing and other objects, features, aspects and advantages of the present invention will become more apparent from the following detailed description of the present invention when taken in conjunction with the accompanying drawings.

### BRIEF DESCRIPTION OF THE DRAWINGS

FIG. 1 is a cross-sectional view showing an embodiment of a cartridge liquid injector of the present invention.

FIG. 2 is a cross-sectional view similar to FIG. 1, showing a state where a cartridge is attached.

FIG. 3 is an enlarged perspective view of a reduction ratio switching portion of the cartridge liquid injector of the present invention.

FIG. 4 is an enlarged perspective view similar to FIG. 3, of the reduction ratio switching portion of the cartridge liquid injector of the present invention.

FIG. 5 is a block diagram of a portion controlling a drive motor of the cartridge liquid injector of the present invention.

### DESCRIPTION OF THE PREFERRED EMBODIMENTS

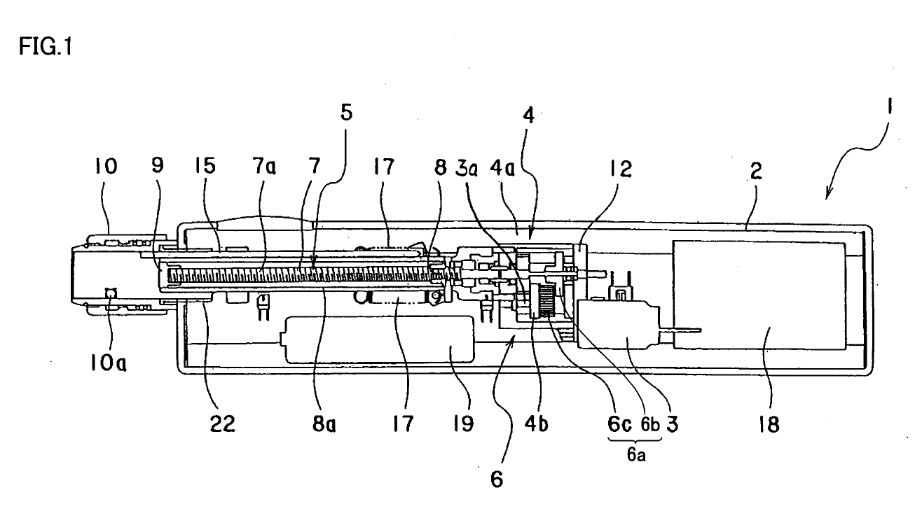

An embodiment of the present invention will be described hereinafter with reference to FIGS. 1 to 5.

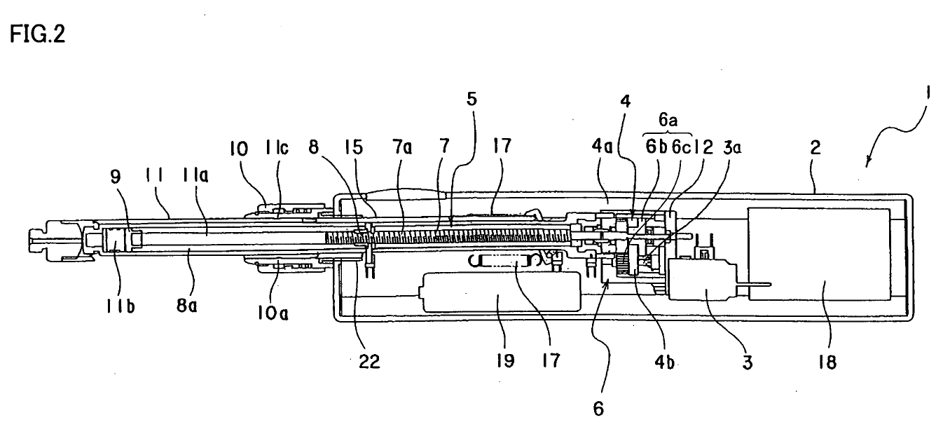

As shown in FIGS. 1 and 2, cartridge liquid injector 1 according to the present embodiment is formed such that an apparatus main body 2 like a long case contains each main component.

In apparatus main body 2, a drive motor 3 implemented by a DC motor or the like rotatable in forward and reverse directions, for applying driving force, is fixed on a base end side. On an output side of drive motor 3, a reduction portion 4 reducing a speed of rotational output of drive motor 3 and a linear motion conversion portion 5 converting rotational output of reduction portion 4 to linear motion are successively coupled.

Reduction portion 4 is implemented in a gear box 12 by a gear group 4a in which a plurality of gears are coupled. A part of gear group 4a serves as a moving gear group 6a movable in an axial direction, that implements a part of a reduction ratio switching portion 6 which will be described later.

An output shaft of gear group 4a serves as a long rotation shaft 7 implementing linear motion conversion portion 5. Rotation shaft 7 is formed to have a length from reduction portion 4 to a tip end of apparatus main body 2. Linear motion conversion portion 5 is formed of a thread portion 7a formed on an outer circumferential surface of rotation shaft 7, a nut 8 screwed thereto, and a sleeve 8a. Though nut 8 is incapable of turning, it is attached movably in the axial direction in such a manner that a direction of movement is switched depending on rotation of rotation shaft 7 in forward and reverse directions.

Pipe-like sleeve 8a in which rotation shaft 7 is loosely inserted is fastened to nut 8 on the outer side in apparatus main body 2. A plunger 9 is fastened to a tip end of sleeve 8a with such a method as screwing.

A cylindrical support element 22 in a cylindrical shape is fastened to a tip end wall portion of apparatus main body 2 in a manner penetrating the tip end wall portion. Cylindrical support element 22 supports an outer circumferential portion of plunger 9 and sleeve 8a in a manner movable in the axial direction. A cylindrical cartridge holder attachment and removal mechanism 10 is fixed to an outer circumferential portion of cylindrical support element 22 concentrically with plunger 9 with such a method as screwing.

As shown in FIG. 1, in a most pulled-back state, a tip end of plunger 9 faces in cartridge holder attachment and removal mechanism 10. As shown in FIG. 2, a cartridge holder 11 in a long cylindrical shape is attached inside cartridge holder attachment and removal mechanism 10.

A cartridge 11a in a long cylindrical shape is inserted in cartridge holder 11. A liquid is hermetically stored in cartridge 11a and an injection plug 11b is provided at a base end portion in a manner movable in the axial direction. Cartridge holder 11 is inserted in cartridge holder attachment and removal mechanism 10 until injection plug 11b of cartridge 11a abuts plunger 9. Cartridge holder 11 is attached to cartridge holder attachment and removal mechanism 10 by engaging a click 10a provided on an inner circumferential surface of cartridge holder attachment and removal mechanism 10 with a recess portion 11c formed on an outer circumferential surface of cartridge holder 11.

When rotation shaft 7 is turned in a forward direction while cartridge 11a and cartridge holder 11 are attached to cartridge holder attachment and removal mechanism 10, plunger 9 is pushed forward by linear motion conversion portion 5. Thus, injection plug 11b of cartridge 11a is moved forward. When rotation shaft 7 is turned in a reverse direction, plunger 9 is pulled back by linear motion conversion portion 5.

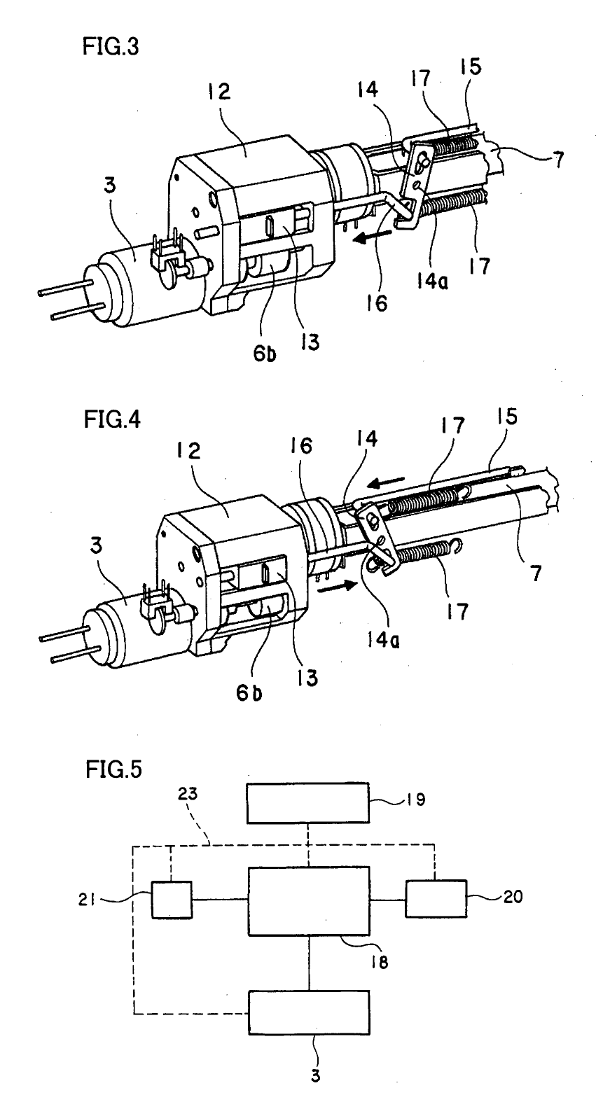

Reduction ratio switching portion 6 serves to switch the reduction ratio such that the reduction ratio of reduction portion 4 in pulling back plunger 9 is lower than that in pushing forward the same. As shown in FIGS. 3 and 4, reduction ratio switching portion 6 is implemented by moving gear group 6a movable in the axial direction by means of moving element 13, that is provided in gear box 12. In the present embodiment shown in FIGS. 1 to 4, moving gear group 6a is formed of two gears 6b and 6c.

As shown in FIGS. 2 and 4, the state where moving element 13 has moved to the tip end side of rotation shaft 7 is the pushed forward state of plunger 9. As gear 6b attached to rotation shaft 7 is engaged with gear 4b attached to a drive shaft 3a located on the drive source side relative to rotation shaft 7, driving force is transmitted.

As shown in FIGS. 1 and 3, the state where moving element 13 has moved to the base end side of rotation shaft 7 is the pulled back state of plunger 9. As a result of movement of moving gear group 6a, gear 6b attached rotation shaft 7 is engaged with gear 6c attached to drive shaft 3a located on the drive source side relative to rotation shaft 7 and driving force is transmitted.

In the present embodiment, the reduction ratio of reduction portion 4 determined by a coupling state of gear groups 4a and 6a, that is, coupling of two gears 6b and 6c, in pulling back is set lower than the reduction ratio of reduction portion 4 determined by a coupling state of gear groups 4a and 6a, that is, coupling of two gears 4b and 6b, in pushing forward.

Reduction ratio switching portion 6 may be formed such that the reduction ratio is automatically switched in response to attachment and removal of cartridge 1 a and cartridge holder 11 to and from cartridge holder attachment and removal mechanism 10 fixed to apparatus main body 2. Specifically, in a position in the rear of gear box 12 shown in FIGS. 1 and 2, a swing link 14 swinging around a swing center 14a is provided on the tip end side of gear box 12. A cartridge detection bar 15 having a length from one end of swing link 14 to the inside of cartridge holder attachment and removal mechanism 10 is provided. A coupling bar 16 couples the other end of swing link 14 and moving element 13 within gear box 12 to each other. As shown in FIG. 3, springs 17 applying elastic force to move swing link 14 in such a direction that moving element 13 is moved to a position in a state where plunger 9 is pulled back are connected to opposing ends of swing link 14.

Moving gear group 6a, that is, two gears 6b and 6c, of reduction ratio switching portion 6 is attached to moving element 13. Therefore, when cartridge 11a and cartridge holder 11 are not attached, as shown in FIGS. 3 and 1, elastic force of spring 17 acts to move swing link 14 and the reduction ratio of reduction portion 4 is set to the reduction ratio in pulling back plunger 9. When cartridge 11a and cartridge holder 11 are attached to cartridge holder attachment and removal mechanism 10, as shown in FIGS. 4 and 2, cartridge detection bar 15 is pushed into apparatus main body 2 against the elastic force of spring 17. Accordingly, swing link 14 and coupling bar 16 are moved and the reduction ratio of reduction portion 4 is set to the reduction ratio in pushing forward plunger 9.

In addition, in the present embodiment, drive motor 3 may be formed such that rotation in forward and reverse directions thereof may be switched in response to attachment and removal of cartridge 11a and cartridge holder 11 to and from cartridge holder attachment and removal mechanism 10. Specifically, as shown in FIG. 5 showing an exemplary block configuration of a control circuit as well as FIGS. 1 and 2, apparatus main body 2 contains a control unit 18 formed with a CPU, a bus and the like, a battery 19, and an attachment and removal sensor 20 detecting a moved state, for example, of moving element 13 and swing link 14 and detecting whether cartridge 11a and cartridge holder 11 are attached to cartridge holder attachment and removal mechanism 10 or not. As attachment and removal sensor 20 detects attachment and removal of cartridge 11a and cartridge holder 11, control unit 18 issues an instruction to cause drive motor 3 to rotate in forward and reverse directions.

Moreover, cartridge liquid injector 1 itself may be turned on and off by using a switch 21. Battery 19 supplies electric power to each portion requiring power supply through an electric line 23.

An operation of the cartridge liquid injector of the present embodiment will now be described.

In a state that cartridge 11a and cartridge holder 11 are not attached to cartridge holder attachment and removal mechanism 10 as shown in FIGS. 1 and 3, cartridge 11a and cartridge holder 11 are pushed into cartridge holder attachment and removal mechanism 10 and attached thereto as shown in FIGS. 2 and 4. Here, cartridge detection bar 15 is pushed into apparatus main body 2 against the elastic force of spring 17. Accordingly, swing link 14 and coupling bar 16 are moved. Moving element 13 is also moved to the tip end side together with moving gear group 6a, and gear 6b attached to rotation shaft 7 is engaged with gear 4b attached to drive shaft 3 a located on the drive source side relative to rotation shaft 7. Thus, the reduction ratio of reduction portion 4 is set to the reduction ratio in pushing forward plunger 9.

When switch 21 is switched on, battery 19 supplies electric power to each portion. Attachment and removal sensor 20 detects attachment of cartridge 11a and cartridge holder 11 to cartridge holder attachment and removal mechanism 10 and outputs such a fact to control unit 18. Control unit 18 issues an instruction to cause drive motor 3 to rotate in the forward direction.

Thus, drive motor 3 rotates in the forward direction. Rotation shaft 7 turns at low speed at the reduction ratio in pushing forward plunger 9, that is set in advance by reduction portion 4. Linear motion conversion portion 5 pushes forward plunger 9. As cartridge 11a and cartridge holder 11 are immovably held by apparatus main body 2 as a result of engagement between click 10a and recess portion 11c with each other, plunger 9 is pushed forward and injection plug 11b moves forward. For example, when a needle (not shown) is attached to the tip end of cartridge 11a, injection plug 11b moves forward to make an injection of a liquid within cartridge 11a. In a state that plunger 9 is pushed forward most, idling of drive motor 3 implemented by a DC motor may be detected, so that rotation of drive motor 3 is stopped.

Thereafter, cartridge 11a and cartridge holder 11 are removed from cartridge holder attachment and removal mechanism 10. Then, as shown in FIG. 3, cartridge detection bar 15 is set to a free state. Swing link 14 is forcibly pivoted by the elastic force of spring 17, and cartridge detection bar 15 returns to the tip end side. Moving element 13 coupled to coupling bar 16 is moved toward the base end side together with moving gear group 6a. Gear 6b attached to rotation shaft 7 is engaged with gear 6c attached to drive shaft 3a located on the drive source side relative to rotation shaft 7. Thus, the reduction ratio of reduction portion 4 is set to the reduction ratio in pulling back plunger 9.

In addition, attachment and removal sensor 20 detects removal of cartridge 1 a and cartridge holder 11 from cartridge holder attachment and removal mechanism 10 and outputs a signal to control unit 18. Control unit 18 issues an instruction to cause drive motor 3 to rotate in the reverse direction. Thus, drive motor 3 rotates in the reverse direction. Rotation shaft 7 turns at high speed at the reduction ratio in pulling back plunger 9, that is set in advance by reduction portion 4. Linear motion conversion portion 5 automatically pulls back plunger 9 at high speed.

In a state that plunger 9 is pulled back most, idling of drive motor 3 implemented by a DC motor may be detected, so that rotation of drive motor 3 is stopped. As plunger 9 is pulled back, cartridge liquid injector 1 returns to a state shown in FIG. 1. Therefore, cartridge holder 11 in which new cartridge 11a is inserted is again attached to cartridge holder attachment and removal mechanism 10, and an operation to move injection plug 11b forward can be repeated.

It is noted that the present invention is not limited to the embodiment above and may be modified as necessary.

For example, an electromagnetic actuator is directly connected to moving element 13, instead of swing link 14, cartridge detection bar 15, coupling bar 16, and spring 17. The electromagnetic actuator may be operated to move moving element 13, depending on an attachment and removal state of cartridge 11a and cartridge holder 11 to and from cartridge holder attachment and removal mechanism 10 detected by attachment and removal sensor 20.

In addition, any reduction portion and reduction ratio switching portion capable of reduction and switching of the reduction ratio by using combination of gears to be engaged may be employed, and therefore, various types of planetary gear mechanisms and gear shift mechanisms may be combined and applied as appropriate.

Although the present invention has been described and illustrated in detail, it is clearly understood that the same is by way of illustration and example only and is not to be taken by way of limitation, the scope of the present invention being interpreted by the terms of the appended claims.

What is claimed is:

1. A cartridge liquid injector, comprising:
a drive motor rotatable in forward and reverse directions, for applying driving force;
a reduction portion reducing a speed of rotational output of said drive motor;
a linear motion conversion portion converting rotational output of said reduction portion to linear motion;
a plunger pushed forward or pulled back by said linear motion conversion portion, for moving forward an injection plug of a cartridge; and
a reduction ratio switching portion switching a reduction ratio of said reduction portion such that the reduction ratio in pulling back said plunger is lower than the reduction ratio in pushing forward said plunger.

2. The cartridge liquid injector according to claim 1, wherein
said reduction ratio switching portion switches the reduction ratio by switching an engaged state of a gear in a reduction gear group forming the reduction portion.

3. The cartridge liquid injector according to claim 1, wherein
said reduction ratio switching portion switches the reduction ratio in response to attachment and removal of the cartridge to and from an injector main body.

4. The cartridge liquid injector according to claim 1, wherein
said drive motor is switched between rotations in forward and reverse directions in response to attachment and removal of the cartridge to and from an injector main body.
